(19) United States
(12) Patent Application Publication
(10) Pub. No.: US 2012/0197309 A1
(43) Pub. Date: Aug. 2, 2012

(54) Bone Anchor Including an Elongate Post With Break-Off Features

(75) Inventor: Bradley E. Steele, Germantown, TN (US)

(73) Assignee: Warsaw Orthopedic, Inc., Warsaw, IN (US)

(21) Appl. No.: 13/016,351

(22) Filed: Jan. 28, 2011

(51) Int. Cl. A61B 17/86; A61B 17/56

(52) U.S. Cl. 606/301; 606/104

(57) ABSTRACT

A bone anchor having a bone engaging portion adapted for anchoring to bone, and an elongate post portion extending from the bone engaging portion and arranged along a longitudinal axis. The elongate post portion includes a proximal end region connected to the bone engaging portion, a break-off region extending axially from the proximal end region, and a guide region extending axially from the break-off region and configured for sliding engagement with an implant, and with the break-off region including a pre-defined fracture initiator zone to facilitate initiation of a fracture therealong upon application of a sufficient torsional force onto the break-off region and removal of a length of the elongate post portion distal of the fracture.

### BACKGROUND

The present invention relates generally to spinal implant systems, and more particularly relates to a bone anchor including an elongate post with break-off features.

Several techniques and systems have been developed for fixing and/or stabilizing the spinal column. In one type of system, a support element such as an elongate spinal rod is disposed longitudinally along a length of the spinal column or along several vertebrae of the spinal column. The spinal rod is typically attached to various vertebrae by way of a number of connectors that connect the spinal rod to a number of bone anchors. A variety of bone anchors can be used to attach the spinal rod to the vertebrae. For example, a bone screw can be threaded into one or more aspects of a vertebra such as, for example, the pedicle region of a vertebra. Additionally, a hook can be wrapped about a portion of a vertebra such as, for example, the lamina region of a vertebra. The bone anchor sometimes includes a connector portion, such as a cylindrical head or shaft, sized for engagement within a corresponding opening in the connector to interconnect the connector with the bone anchor. In some instances, the connector and/or the elongate spinal rod positioned within a rod receiving passage in the connector is reduced into engagement with the post/shaft via guided displacement along an elongate post that forms an integral part of the cylindrical head/shaft of the bone anchor. After reduction is complete and during the surgical procedure, the excess portion of the post/shaft is cut off via a post cutter or another type of shearing device. Conventional post cutters rely on lever arms that give the surgeon a mechanical advantage to cut the post via a cutting or shearing action. However, the force applied to the post cutter to effectuate the cutting action is relatively large, and the cutter instrument can be large and obstructive and is often difficult to manipulate and control during the cutting process.

Thus, there remains a need for a bone anchor including an elongate post that does not require the use of a conventional post cutter instrument to remove the excess portion of the post. The present invention satisfies this need and provides other benefits and advantages in a novel and unobvious manner.

### SUMMARY

While the actual nature of the invention covered herein can only be determined with reference to the claims appended hereto, certain forms of the invention that are characteristic of the preferred embodiments disclosed herein are described briefly as follows.

In one form of the present invention, a bone anchor is provided having a bone engaging portion adapted for anchoring to bone, and an elongate post portion extending from the bone engaging portion and arranged along a longitudinal axis. The elongate post portion includes a proximal end region connected to the bone engaging portion, a break-off region extending axially from the proximal end region, and a guide and reduction region extending axially from the break-off region and configured for sliding engagement with an implant, and with the break-off region including a pre-defined fracture initiator zone to facilitate initiation of a fracture therealong upon application of a sufficient torsional force onto the break-off region and removal of a length of the elongate post portion distal of the fracture.

In another form of the present invention, a bone anchor is provided having a bone engaging portion adapted for anchoring to bone, and an elongate post portion extending from the bone engaging portion and arranged along a longitudinal axis. The elongate post portion includes a proximal end region connected to the bone engaging portion, a break-off region extending axially from the proximal end region, and a guide region extending axially from the break-off region and configured for sliding engagement with an implant. The break-off region includes a plurality of annular grooves extending about the longitudinal axis and axially offset from one another along the longitudinal axis, the annular grooves facilitating initiation of a fracture along a select one of the annular grooves upon application of a sufficient torsional force onto the break-off region and removal of a length of the elongate post portion distal of the fracture. The break-off region further includes one or more tool engagement features positioned proximately adjacent the annular grooves, the tool engagement features configured for engagement by a torque application instrument to apply the sufficient torsional force onto the break-off region to facilitate initiation of the fracture along the select one of the annular grooves.

It is one object of the present invention to provide a bone anchor including an elongate post with break-off features. Further embodiments, forms, features, aspects, benefits, objects, and advantages of the present application will become apparent from the detailed description and figures provided herewith.

### BRIEF DESCRIPTION OF THE DRAWINGS

FIG. 1 is a perspective view of a bone anchor according to one form of the present invention.

FIG. 2 is a first cross sectional view of the bone anchor illustrated in FIG. 1.

FIG. 2a is an enlarged break-off region of the bone anchor illustrated in FIG. 2.

FIG. 3 is a second cross sectional view of the bone anchor illustrated in FIG. 1.

FIG. 3a is an enlarged break-off region of the bone anchor illustrated in FIG. 3.

FIG. 4 is a perspective view of a spinal implant system according to one embodiment for use with the bone anchor illustrated in FIG. 1, as shown in a first operational configuration.

FIG. 5 is a perspective view of the spinal implant system, as shown in a second operational configuration.

FIG. 6 is a perspective view of the spinal implant system, as shown in a third operational configuration.

### DESCRIPTION OF THE ILLUSTRATED EMBODIMENTS

For the purpose of promoting an understanding of the principles of the present invention, reference will now be made to the embodiments illustrated in the drawings and specific language will be used to describe the same. It will nevertheless be understood that no limitation of the scope of the invention is hereby intended. Any alterations and further modifications in the described embodiments, and any further applications of the principles of the invention as described herein are contemplated as would normally occur to one skilled in the art to which the invention relates.

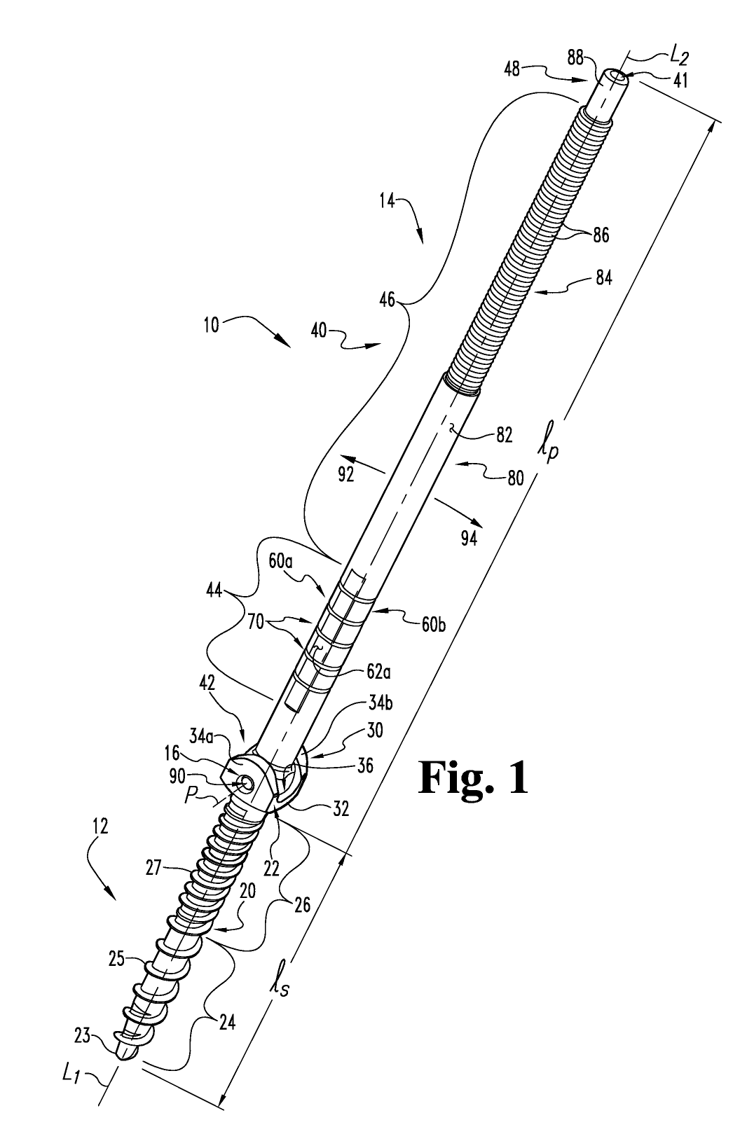

Referring collectively to FIGS. 1-3, shown therein is a bone anchor 10 according to one form of the present invention. The bone anchor 10 generally includes a distal bone engaging portion 12 and a proximal connecting portion 14. In the illustrated embodiment, the proximal connecting portion 14 is pivotally engaged to the distal bone engaging portion 12 via a pivot mechanism 16 to permit pivotal movement of the proximal connecting portion 14 relative to the distal bone engaging portion 12 about a pivot axis P. However, it should be understood that other embodiments are also contemplated where the distal bone engaging portion 12 is rigidly connected to the proximal connecting portion 14, and still other embodiments where the distal bone engaging portion 12 is unitarily integral with the proximal connecting portion 14 so as to define a single-piece monolithic bone anchor 10.

In the illustrated embodiment, the bone anchor 10 is configured as a bone screw, and more particularly as a pedicle bone screw, with the bone engaging portion 12 adapted for anchoring in bone and including a threaded shank 20 and a head 22, each extending generally along a longitudinal axis L₁. In one embodiment, the threaded shank 20 includes a distal tip 23 configured to penetrate bone. In the illustrated embodiment, the distal tip 23 is tapered or pointed to facilitate entry into bone. However, in other embodiments, the distal tip 23 may define a blunt or rounded end. In further embodiments, the distal tip 23 or other portions of the distal end portion of the threaded shank 20 may be provided with one or more cutting edges or flutes (not shown) to provide the threaded shank 20 with self-cutting or self-tapping capabilities. In still other embodiments, the threaded shank 20 may be provided with an axial passage (not shown) extending partially or entirely therethrough to define a cannulation opening, and may be further provided with transverse passages that communicate with the axial passage to define fenestration openings. The cannulation and fenestration openings may be used to deliver a material such as, for example, bone cement through the threaded shank 20 and into areas of the bone axially or laterally adjacent the distal end portion or other portions of the threaded shank 20. Additionally, in the illustrated embodiment, the threaded shank 20 has an overall shank length lₛ and defines a first threaded section 24 extending along a first portion of the shank length lₛ from the distal end toward the proximal end, and a second threaded section 26 extending contiguously from the first threaded section 24 to the proximal end along a second portion of the shank length lₛ. The first threaded section 24 includes a first threading 25 that is adapted for anchoring in the cancellous region of a bone, and the second threaded section 26 includes a relatively finer threading 27 that is adapted for anchoring in the cortical region of the bone.

In one embodiment, the length of the first threaded section 24 extends along at least about one half of the overall length lₛ of the threaded shank 20, with the length of the second threaded section 26 extending along the remainder of the overall shank length lₛ. However, it should be understood that other lengths of the first threaded section 24 relative to the overall shank length lₛ are also contemplated as falling within the scope of the present invention. As should be appreciated, the particular ratio between the lengths of the first and second threaded sections 24, 26 may be selected based on the characteristics of the bone to which the threaded shank 20 is to be engaged. As discussed above, the first threaded section 24 includes thread features that are particularly suited for anchoring in the cancellous region of bone, and the second threaded section 26 includes thread features that are particularly suited for anchoring in the cortical region of bone. In order to maximize the anchoring effectiveness of the threaded shank 20, the lengths of the first and second threaded sections 24, 26 should preferably correspond to the desired anchoring depth within the cancellous region of bone and the thickness of the cortical region of bone, respectively.

In the illustrated embodiment, the first threading 25 includes a single thread lead in the form of a helical thread pattern which defines the first threaded section 24, and the second threading 27 cooperates with the first threading 25 to provide a dual lead in the form of double helical thread pattern which defines the second threaded section 26. In the illustrated embodiment, the first and second threadings or thread leads 25, 27 are provided in the form of a helix that extends substantially continuously about the longitudinal axis L₁ and substantially entirely along the overall length lₛ of the threaded shank 20. Because the second threading 27 is preferably uniformly and centrally offset or interleaved with the first threading 25, the threadings 25, 27 appear to spiral together along the length of the second threaded section 26 as a continuous thread, but which in actuality comprise separate threadings. Providing separate threadings along the second threaded section 26 allows the thread pitch associated with each of the first and second threaded sections 24, 26 to be equal if so desired. Further details of a bone screw including a multi-lead threaded shank suitable for use in association with the present invention can be found in commonly owned U.S. Patent Application Publication No. 2007/0233122 to Denis et al., the contents of which are incorporated herein by references in their entirety. However, it should be understood that other types of bone screws including other configurations of threaded shanks may be used in association with the present invention.

In the illustrated embodiment, the head 22 is unitarily integral with the threaded shank 20 to provide the bone engaging portion 12 of the bone anchor 10 as a monolithic single-piece structure. However, other embodiments are also contemplated wherein the head 22 and the threaded shank 20 constitute separate/individual elements that may be assembled together to define the bone engaging portion 12. In one embodiment, the head 22 may be rotationally/pivotally attached to the threaded shank 20 so as to allow the head 22 to pivot and/or rotate relative to the threaded shank 20 to thereby provide the bone engaging portion 12 with poly-axial or multi-axial capabilities. One example of a poly-axial or multi-axial bone screw is disclosed in commonly owned U.S. Pat. No. 5,879,350 to Sherman et al., the contents of which are hereby incorporated by reference in their entirety. Although a particular type and configuration of the bone engaging portion 12 of the bone anchor 10 has been illustrated and described herein, it should be understood that other types and configurations of bone engaging portions are also contemplated for use in association with the present invention.

In one particular embodiment, the head 22 is configured as a yoke or clevis 30 including a transverse base or bottom portion 32 and a pair of leg portions 34a, 34b extending axially from the base portion 32 and arranged generally parallel with one another along the longitudinal axis L₁ and defining a space or U-shaped channel 36 therebetween. In the illustrated embodiment, the base portion 32 defines a spherical shaped concave bottom surface 33, and the leg portions 34a, 34b define a pair of opposing substantially flat/planar surfaces 35a, 35b facing the channel 36. The leg portions 34a, 34b further define a pair of aligned openings 37a, 37b arranged generally along the pivot axis P and sized for receipt of a pivot pin 90 to permit pivotal movement of the proximal connecting portion 14 relative to the bone engaging portion 12 of the bone anchor 10. The leg portions 34a, 34b or other portions of the head 22 may also be provided with tool engaging features configured for releasable engagement with a driver instrument (not shown) to drive the distal bone engaging portion 12 of the bone anchor 10 into bone and/or to remove the distal bone engaging portion 12 of the bone anchor 10 from bone. The head 22 may also be provided with a friction member 38 including a lower portion 38a positioned within a recess extending toward the threaded shank 20 and an upper portion 38b extending into the space 36 between the leg portions 34a, 34b. The friction member 38 is also provided with an annular flange 39 positionable within a corresponding groove defined in the recess to maintain the friction member 38 in position relative to the head 22. The friction member 38 may be formed of a flexibly elastic and/or resilient material to provide a degree of control over the pivotal movement of the proximal connecting portion 14 relative to the bone engaging portion 12.

Although the distal bone engaging portion 12 of the bone anchor 10 has been illustrated and described as being configured as a bone screw having a particular thread configuration and a particular head, it should be understood that other types/configurations of threaded shanks having different thread configurations and/or other types of heads are also contemplated. Additionally, it should be understood that other types and configurations of the distal bone engaging portion 12 are also contemplated including, for example, hooks, pins, bolts, clamps, staples, interbody devices, or any other type of bone anchor device know to those having ordinary skill in the art.

In the illustrated embodiment, the proximal connecting portion 14 is configured as an elongate post 40 having an overall post length lₚ extending generally along a longitudinal axis L₂. In one embodiment, the elongate post 40 has a generally circular outer cross section. However, other cross sectional configurations are also contemplated. In one embodiment, the proximal connecting portion 14 is pivotally connected to the bone engaging portion 12 via the pivot mechanism 16 to allow relative pivotal movement therebetween about the pivot axis P. In one specific embodiment, the overall length lₚ of the elongate post 40 is greater than the overall length lₛ of the threaded shank 20. In another specific embodiment, the overall length lₚ of the elongate post 40 is greater than twice the overall length lₛ of the threaded shank 20. In a further specific embodiment, the overall length lₚ of the elongate post 40 is approximately three times greater than the overall length lₛ of the threaded shank 20. However, it should be understood that other embodiments are also contemplated wherein the overall length lₚ of the elongate post 40 is approximately equal to the overall length lₛ of the threaded shank 20. It is also contemplated that in some embodiments, the overall length lₚ of the elongate post 40 may be somewhat less than the overall length lₛ of the threaded shank 20.

In the illustrated embodiment, the elongate post 40 generally includes a proximal end region 42, a break-off region 44, a guide region 46, and a distal end region 48, and may also be provided with a cannulated opening or passage 41 extending therethrough and arranged generally along the longitudinal axis L₂. In the illustrated embodiment, the cannulated opening or passage 41 extends entirely through the elongate post 40. However, in other embodiments, the cannulated opening or passage 41 may extend along less than the entire length of the elongate post 40. In one such embodiment, the cannulated opening or passage 41 may be substantially limited to the proximal end region 42 and the break-off region 44 of the elongate post 40. In still further embodiments, the elongate post 40 need not include the cannulated opening or passage 41, but may instead define a solid/non-cannulated post.

In the illustrated embodiment, the proximal end region 42 of the elongate post 40 has a spherical shaped portion 52 and a circular cylindrical shaped portion 54 extending axially and distally from the spherical shaped portion 52. The spherical shaped portion 52 includes a proximally facing convex outer surface 53 that substantially corresponds in size and shape to the concave bottom surface 33 defined by the base portion 32 of the bone anchor head 22, and with the proximally facing convex outer surface 53 positioned proximately adjacent the concave bottom surface 33 to permit pivotal movement of the proximal connecting portion 14 relative to the distal bone engaging portion 12 about the pivot axis P. The spherical shaped portion 52 also includes a pair of flattened or truncated surfaces 55a, 55b that are positioned proximately adjacent the substantially flat/planar surfaces 35a, 35b defined by the leg portions 34a, 34b of the bone anchor head 22. The spherical shaped portion 52 further defines a proximal portion of the cannulated opening or passage 41 that is sized and shaped for receipt of the upper portion 38b of the friction member 38, and also defines an opening 57 extending therethrough and arranged generally along the pivot axis P. The opening 57 in the spherical shaped portion 52 is positioned in general alignment with the pair of aligned openings 37a, 37b in the leg portions 34a, 34b of the bone anchor head 22 for receipt of the pivot pin 90 therein to thereby pivotally attach the proximal connecting portion 14 to the distal bone engaging portion 12 to provide for pivotal movement therebetween about the pivot axis P. The circular cylindrical shaped portion 54 of the proximal end region 42 of the elongate post 40 defines a substantially smooth circular outer surface 58.

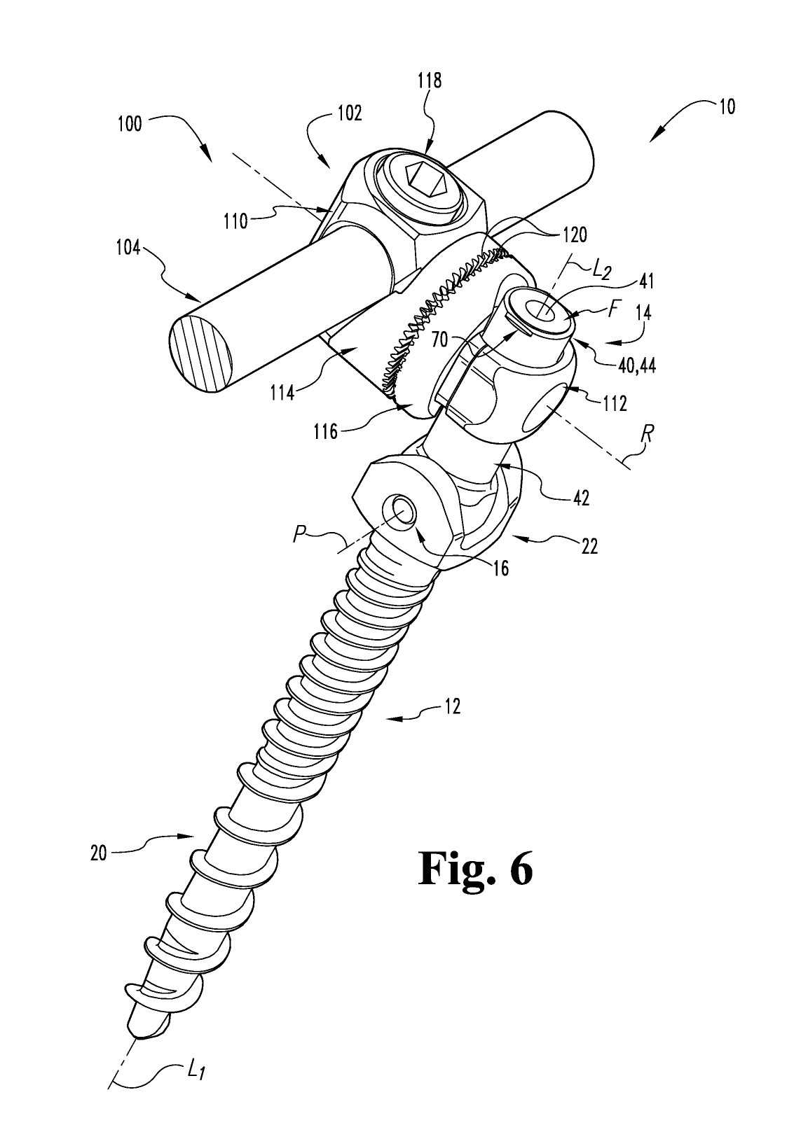

The break-off region 44 of the elongate post 40 extends axially and distally away from the proximal end region 42. In the illustrated embodiment, the break-off region 44 has a substantially cylindrical configuration defining opposite circular portions 60a, 60b that generally correspond in size and shape to the circular outer surface 58 defined by the cylindrical shaped portion 54 of the proximal end region 42. The break-off region 44 further defines a pair of opposite flattened or truncated portions 62a, 62b positioned on opposite sides of the break-off region 44. The circular portions 60a, 60b and the flattened or truncated portions 62a, 62b together define a pair of opposite circular surfaces 64a, 64b and a pair of flat/planar surface 66a, 66b extending from the circular surface 64a to the opposite circular surface 64b. The break-off region 44 also defines a series of pre-defined fracture initiator zones 70 axially offset from one another along the longitudinal axis L₂. The pre-defined fracture initiator zones 70 are configured to facilitate initiation of a fracture along a select one of the zones 70 upon application of a sufficient torsional force onto said break-off region 44 so as to allow for removal of a length of the elongate post 40 distal of the fracture zone F (FIG. 6).

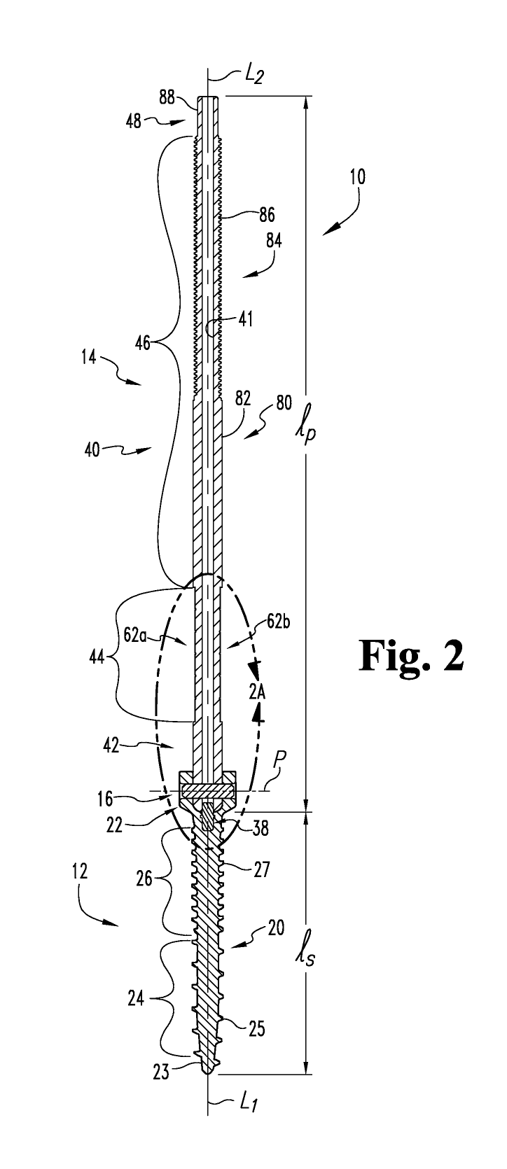

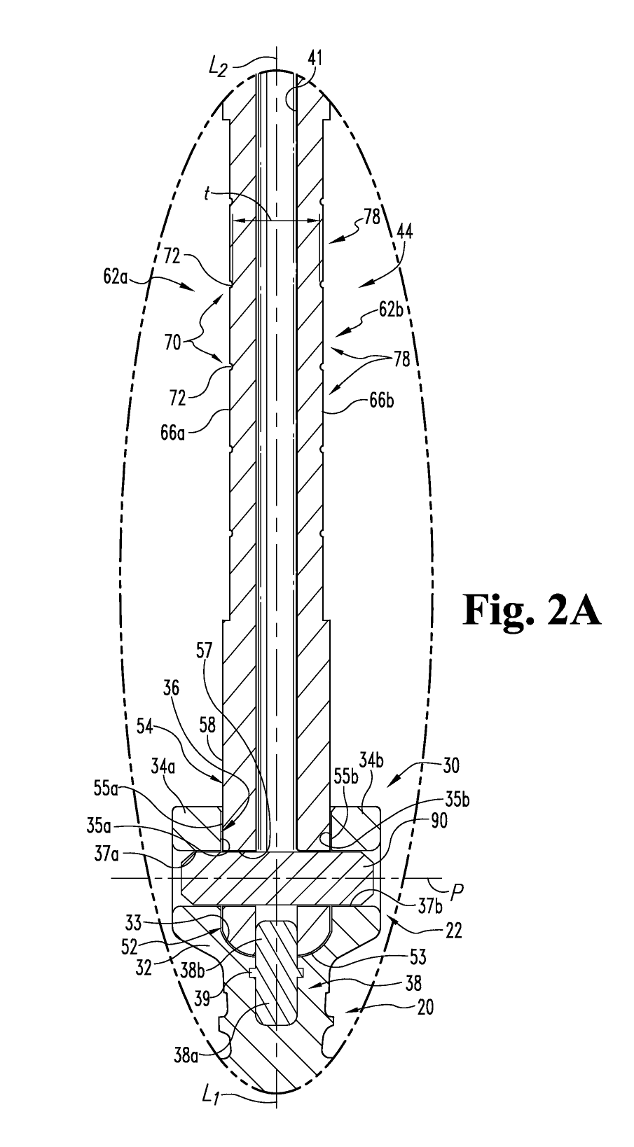

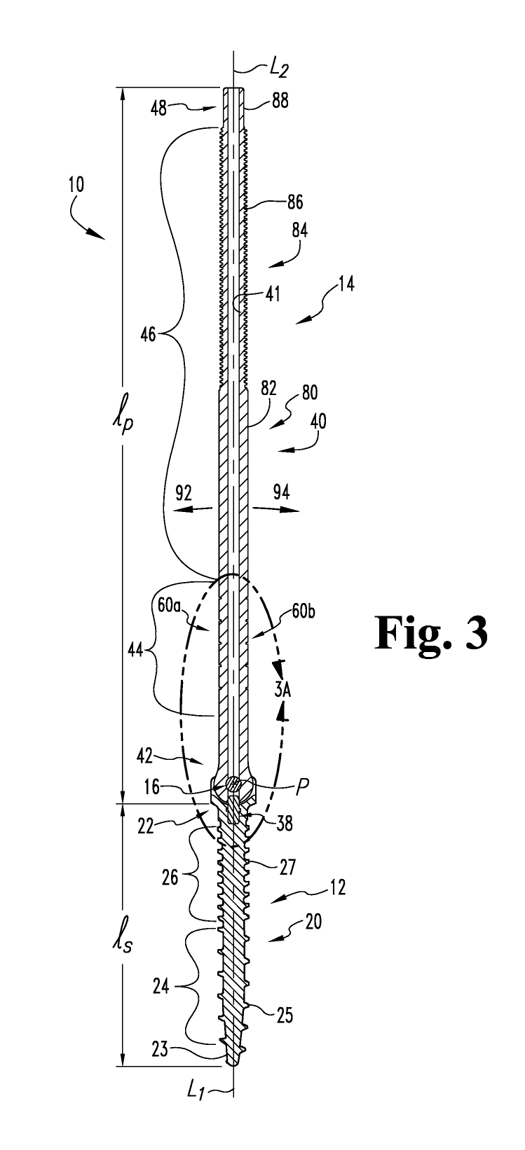

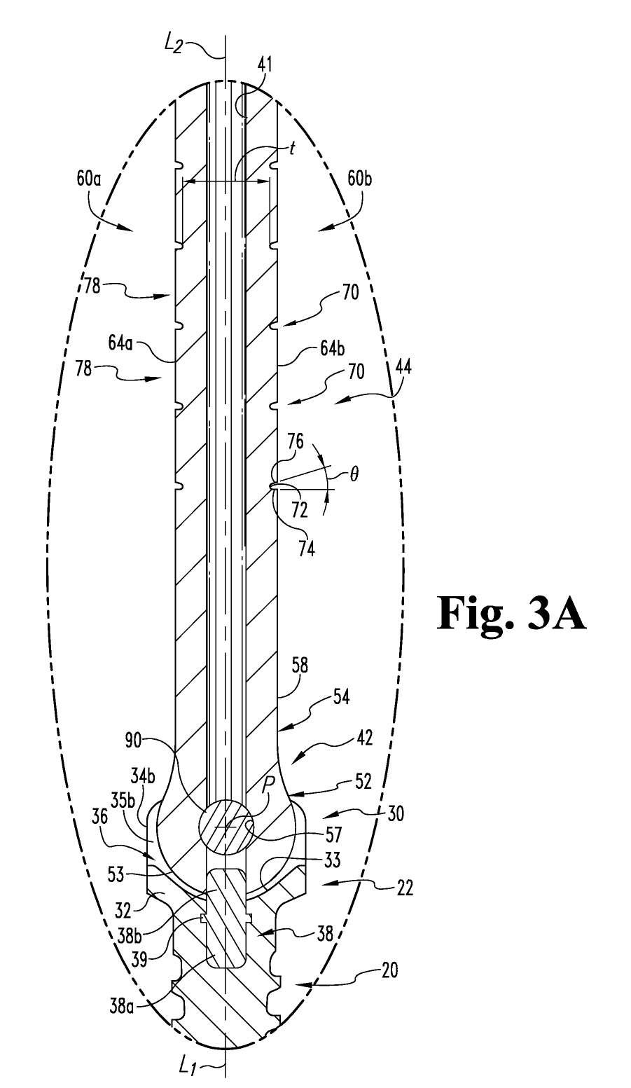

In the illustrated embodiment, the fracture initiator zones 70 are configured as circumferential or annular grooves or scored areas extending about the outer perimeter of the break-off region 44, axially offset from one another along the longitudinal axis L₂, and extending inwardly into the circular outer surfaces 64a, 64b and the flat/planar surface 66a, 66b. As illustrated in FIG. 3A, along the circular portions 60a, 60b of the break-off region 44, each of the annular grooves 70 has an at least partially arcuate configuration including a concave bottom surface 72 and a pair of generally flat/planar side surfaces 74, 76 extending outwardly from the concave bottom surface 72 to the circular outer surfaces 64a, 64b of the circular portions 60a, 60b. The lower or proximal side surface 74 is arranged generally perpendicular or normal to the longitudinal axis L₂, whereas the upper or distal side surface 76 is outwardly tapered relative to the lower side surface 74 at an oblique taper angle θ. In one embodiment, the oblique taper angle θ falls within a range of approximately 5 degrees to 15 degrees. However, other taper angle θ are also contemplated. Since the truncated portions 62a, 62b provide the break-off region 44 with a reduced outer dimension relative to the circular portions 60a, 60b, as illustrated in FIG. 2A, the annular grooves 70 extend into the flat/planar surfaces 66a, 66b along the truncated portions 62a, 62b at a reduced groove depth, and therefore do not define the entire inner profile of the annular grooves 70 defined by the circular portions 60a, 60b.

As should be appreciated, the series of fracture initiator zones or grooves 70 each constitute a reduced strength or frangible portion of the break-off region 44. More specifically, the grooves 70 provide the break-off region 44 with regions of reduced strength relative to axially adjacent portions 78 of the break-off region 44 to thereby provide pre-defined fracture initiators or break zones along the break-off region 44. As should be appreciated, application of a rotational force or torque (or application of a bending or shear force) to the elongate post 40 above a threshold level will cause the elongate post 40 to fracture or break along a select one of the grooves 70, thereby allowing selective separation and removal of a length of the elongate post 40 distal of the fracture/break zone F (FIG. 6). In the illustrated embodiment, the break-off region 44 includes five of the fracture initiator zones or grooves 70. However, it should be understood that the break-off region 44 may be provided with any number of the fracture initiator zones or grooves 70, including one, two, three, four or six or more of the fracture initiator zones or grooves 70.

As illustrated in FIGS. 2A and 3A, in one embodiment, the grooves 70 provide the break-off region 44 with a series of reduced transverse cross sections relative to the axially adjacent portions 78 to thereby provide the pre-defined fracture initiators or break zones. More specifically, the grooves 70 provide the break-off region 44 with a series of reduced transverse cross sections via localized reductions in the material thickness t of the wall of the break-off region 44. Although the illustrated embodiment of the grooves 70 comprises annular grooves extending about the exterior surface of the break-off region 44, it should be understood that in other embodiments, the grooves 70 may be provided along an interior surface of the break-off region 44 (i.e., about the inner surface defined by the cannulated opening 41). Additionally, although the grooves 70 are illustrated as extending about the entire outer perimeter of the break-off region 44 (i.e., about both the circular portions 60a, 60b and the truncated portions 62a, 62b), it should be understood that in other embodiments, the grooves 70 may extend about only a portion of the outer perimeter of the break-off region 44 (i.e., about only the circular portions 60a, 60b or about only the truncated portions 62a, 62b).

As should also be appreciated, the flattened or truncated portions 62a, 62b of the break-off region 44 provide the elongate post 40 with tool engaging features positioned proximally adjacent the series of fracture initiator zones or grooves 70. The flattened or truncated portions 62a, 62b are configured for releasable engagement with a driver or torque application instrument (not shown), such as a wrench or another type of driver instrument, to facilitate application of a rotational force/torque onto the elongate post 40 to drive the distal bone engaging portion 12 of the bone anchor 10 into bone and/or to initiate breaking or fracturing of the elongate post 40 along a select one of the grooves 70. The flattened or truncated portions 62a, 62b may also serve as a point of releasable engagement with a counter torque instrument such that the rotational force/torque applied to the elongate post does not translate to the bone engaging region 12. Additionally, it should be understood that a bending force may be applied to the elongate post 40 to initiate breaking or fracturing along a select one of the grooves 70 formed along the break-off region 44. Although the flattened or truncated portions 62a, 62b have been illustrated as extending along the break-off region 44, it should be understood that the flattened or truncated portions 62a, 62b may be defined by other portions of the elongate post 40, including the guide region 46 or the distal end region 48. It should also be understood that the elongate post 40 may be provided with other types and configurations of tool engaging features other than flattened/truncated regions. For example, one or more portions of the elongate post 40 may be provided with a non-circular outer transverse cross section having a hexagonal configuration or a Torx-shaped configuration to facilitate releasable engagement with a driver or torque application instrument. Other shapes and configurations are also contemplated including, for example, a star-shaped configuration, a cross-shaped configuration, a slot-shaped configuration, other non-circular or polygonal shapes and configurations, or a series of openings or apertures extending into the outer surface of the elongate post 40 to facilitate releasable engagement with a driver or torque application instrument.

The guide region 46 of the elongate past 40 extends axially and distally from the break-off region 44. In the illustrated embodiment, the guide region 46 has a substantially cylindrical configuration and includes a first portion 80 defining a substantially smooth circular outer surface 82, and a second portion 84 defining external threads 86. As should be appreciated, the guide region 46 may be used to guide implants or other devices axially along the elongate post 40 toward the head 24 of the bone engaging portion 12, and the external threads 86 may be used to threadingly engage and axially advance an instrument or device (such as a sleeve or nut) along the elongate post 40 to advance or reduce an implant or device toward the bone engaging portion 12 of the bone anchor 10. The distal end region 48 of the elongate past 40 extends axially and distally from the guide region 46 and has a circular cylindrical configuration defining a substantially smooth circular outer surface 88 having a diameter substantially corresponding to the inner thread root diameter of the external threads 86. The distal end region 48 is configured to facilitate loading of implants or other devices or instruments onto the elongate post 40.

In the illustrated embodiment, the pivot mechanism 16 is configured as a pivot pin 90 extending along the pivot axis P and through the opening 57 in the proximal end region 42 of the elongate post 40 and into the openings 37a, 37b in the leg portions 34a, 34b of the bone anchor head 22. The pivot pin 90 serves to pivotally attach the proximal connecting portion 14 to the distal bone engaging portion 12 to allow for pivotal movement of the proximal connecting portion 14 relative to the distal bone engaging portion 12 about the pivot axis P in the direction of arrows 92, 94. In the illustrated embodiment, the pivot pin 90 has a generally circular outer surface defining an outer diameter sized in relatively close tolerance with the inner diameter of the openings 37a, 37b and 57. However, it should be understood that other types and configurations of the pivot mechanism 16 and/or the pivot pin 90 are also contemplated as falling within the scope of the present invention. It should also be understood that other embodiments of the bone anchor 10 are contemplated where the proximal connecting portion 14 is rigidly and non-pivotally attached to the distal bone engaging portion 12.

The components of the bone anchor 10, including the distal bone engaging portion 12 and the proximal connecting portion 14, may be formed of any suitable biocompatible material such as, for example, titanium, a titanium alloy, stainless steel, metallic alloys, non-metallic materials, or other materials known to those of skill in the art that possess the mechanical and biocompatible properties suitable for implantation within the body and attachment to bone.

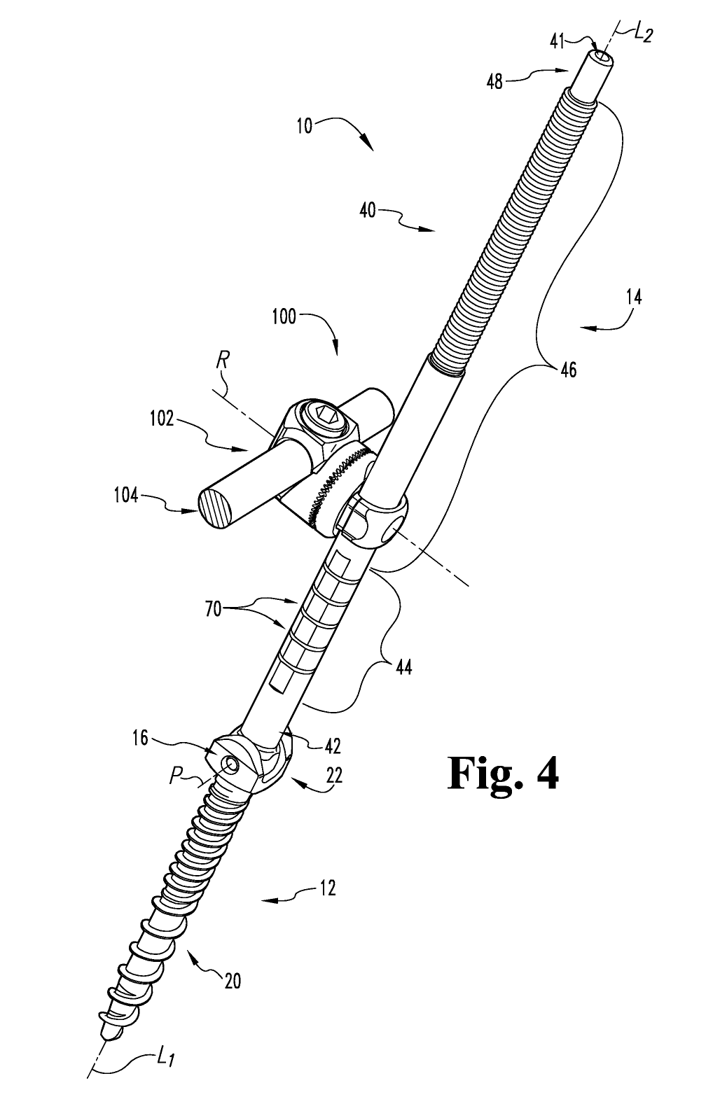

Referring to FIGS. 4-6, illustrated therein is one embodiment of a spinal implant assembly 100 with which the bone anchor 10 may be used. In one embodiment, the spinal implant assembly 100 generally includes the bone anchor 10, an adjustable connector assembly 102, and an elongate support member 104 that is interconnected with the bone anchor 10 via the spinal connector assembly 100. In the illustrated embodiment the connector assembly 102 extends generally along a rotational axis R and is configured to transversely interconnect the elongate support member 104 with the proximal connecting portion 14 of the bone anchor member 10.

In one embodiment, the bone anchor 10 comprises a bone screw and the elongate support member 104 comprises a spinal rod. However, other types and configurations of the bone anchor 10 and the elongate support 104 are also contemplated for use in association with the present invention. Additionally, it should be understood that the connector assembly 102 may be used to interconnect various types and configurations of spinal implants or devices, and is not limited to interconnecting the bone screw 10 with a spinal rod 104. For example, the connector assembly 102 may also be used to interconnect other types and configurations of elongate members with other types and configurations of bone anchors. It should also be understood that the bone anchor 10 and the implant assembly 100 may be used in fields outside of the spinal field including, for example, in fixation or stabilization systems that are attached to other bony structures including the pelvis, the skull and/or the occiput, long bones, or other bony structures that would occur to those having ordinary skill in the art.

In the illustrated embodiment, the elongate support member or spinal rod 104 includes a substantially smooth outer surface defining a circular outer cross section having a substantially uniform outer diameter. However, it should be understood that the elongate spinal rod 104 may be provided with other cross sectional shapes, and the outer surface may be roughened (e.g., via knurling or threading) or otherwise textured to facilitate secure connection with the connector assembly 102. It should also be understood that other types and configurations of elongate support members are also contemplated for use in association with the present invention including, for example, bars, elongate plates, wires, tethers, or any other type of elongate support member know to those having ordinary skill in the art.

As shown most clearly in FIG. 6, in the illustrated embodiment, the connector assembly 102 generally includes a first connector member 110 configured for coupling with the elongate support member 104, a second connector member 112 configured for coupling with the proximal connecting portion 14 of the bone anchor member 10, a first washer member 114 associated with the first connector member 110, a second washer member 116 associated with the second connector member 112, and including a set screw member 118 threadedly engaged with the first connector member 110 to capture the elongate support member 104 within a passage in the first connector member 110. The first and second washer members 114, 116 include interdigitating or intermeshing spline elements 120 configured to aid in selectively preventing relative rotational movement between the first and second connector members 110, 112 about the rotational axis R.

The first connector member 110 includes a first passage sized and configured to receive the elongate support member 104 therein, and a threaded opening in communication with the first passage and configured for threading receipt of the set screw member 118. The second connector member 112 includes a second passage sized and configured to receive the proximal connecting portion 14 of the bone anchor 10 therein. The connector assembly 102 is configured such that the first and second connector members 110, 112 are rotationally engaged to one another in a manner allowing relative rotational movement between the first and second connector members 110, 112 about the rotation axis R. As should be appreciated, the angular orientation of the elongate support member 104 may be adjusted relative to the proximal connection portion 14 of the bone anchor 10 to a desired angular orientation via rotation of the first connector member 110 relative to the second connector member 112 about the rotational axis R.

Once the select angular orientation between the elongate support member 104 and the proximal connection portion 14 of the bone anchor 10 has been achieved, the set screw member 118 is advanced along the threaded opening in the first connector member 110 and into compressed engagement with the elongate support member 104. The set screw member 118 urges the elongate support member 104 into abutting engagement against an engagement surface of the first washer member 114, which results in axial displacement of the first washer member 114 into engagement with the second washer member 116, which in turn axially displaces the second washer member 116 toward the second connector member 112 and into compressed engagement with the proximal connecting portion 14 of the bone anchor 10 positioned within the passage in the second connector member 112.

Threading the set screw member 118 along the threaded opening in the first connector member 110 serves multiple functions. First, tightening the set screw member 118 against the elongate support member 104 compresses the elongate support member 104 into abutting engagement against the engagement surface of the first washer member 114 to thereby prevent further axial or rotational movement of the elongate support member 104 within the first passage of the first connector member 110. Second, tightening the set screw member 118 also compresses the spline elements 120 defined by the first and second washer members 114, 116 into intermeshing or interdigitating engagement with one another, which in turn selectively prevents relative rotational movement between the washer members 114, 116 and relative rotational movement between the first and second connector members 110, 112, thereby locking the elongate support member 104 and the proximal connection portion 14 of the bone anchor 10 at a select angular orientation relative to one another. Third, tightening the set screw member 118 also compresses an outer surface of the second washer member 116 against the proximal connecting portion 14 of the bone anchor 10 positioned within the second passage in the second connector member 112, which in turn compressingly engages the proximal connecting portion 14 of the bone anchor 10 against an inner surface defined by the second passage to substantially prevent further axial or rotational movement of the bone anchor 10 relative to the second connector member 112. Accordingly, a single set screw member 118 may be used to secure the elongate support member 104 and the bone anchor 10 within the respective passages in the connector members 110, 112, and to lock the connector members 110, 112 at a select rotational position relative to one another about the rotational axis R, which in turn locks the elongate support member 104 and the proximal connecting portion 14 of the bone anchor 10 at a select angular orientation relative to one another.

Further details regarding the connector assembly 102 can be found in commonly owned U.S. patent application Ser. No. 12/846,298 to Rezach, the contents of which are incorporated herein by references in their entirety. However, it should be understood that other types and configurations of connector assemblies are also contemplated for use in association with the present invention.

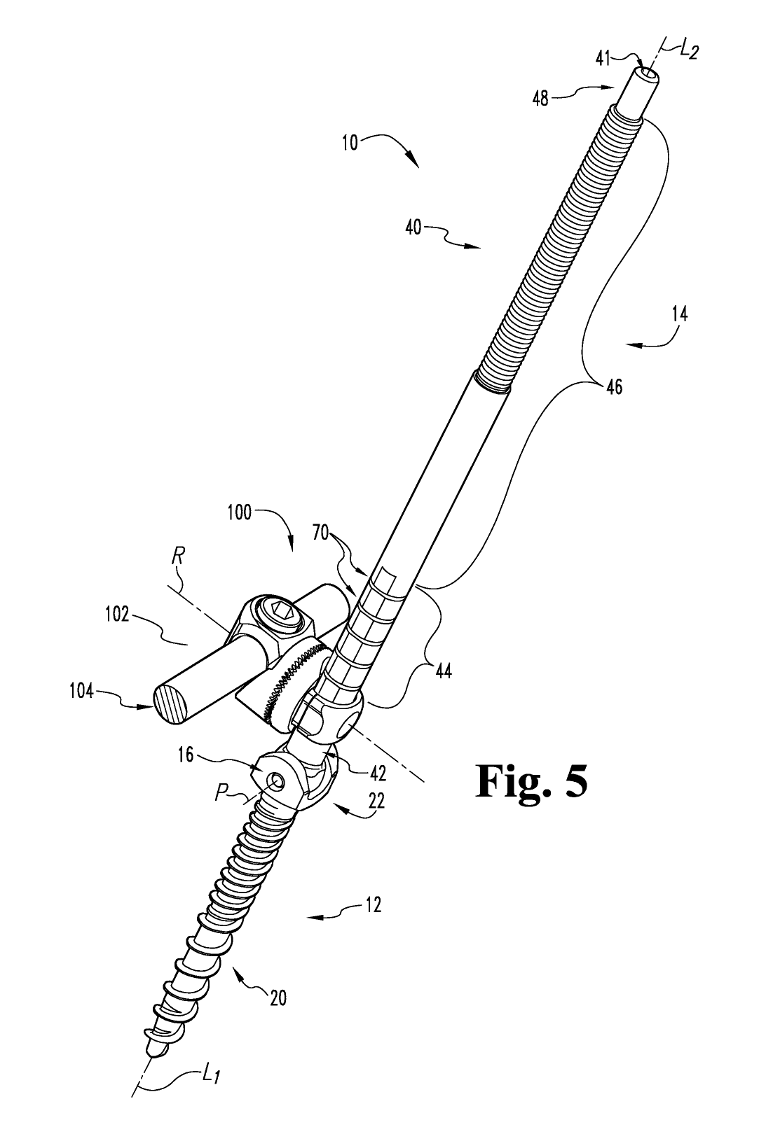

As shown in FIG. 4, the adjustable connector assembly 102, either with or without the elongate support member 104 engaged therewith, may be loaded onto the distal end region 48 of the elongate post 40 and axially guided along the guide region 46 toward the bone engaging portion 12 of the bone anchor 10. As shown in FIG. 5, the adjustable connector assembly 102 may be further advanced onto the break-off region 48 of the elongate post 40 and axially beyond at least one of the fracture initiator zones or grooves 70. At this point, if not already in position, the elongate support member 104 may be inserted into the passage in the first connector member 110 of the connector assembly 102, and the first connector member 110 (and the elongate support member 104) may be rotationally adjusted relative to the second connector member (and the elongate post 40) to a desired angular orientation. The set screw 118 is then tightened to lock the connector assembly 102 at the desired angular orientation and position, as well as to lock the elongate support member 104 and the elongate post 40 within the passages in the connector members 110, 112, respectively. As shown in FIG. 6, application of a rotational force/torque onto the elongate post 40, and more specifically onto the break-off region 44 via engagement of a driver or torque application instrument (not shown) with the flattened or truncated portions 62a, 62b, will initiate breaking or fracturing of the elongate post 40 along a fracture/break zone F corresponding to a select one of the fracture initiator zones or grooves 70. The breaking or fracturing of the break-off region 44 along the fracture zone F allows for selective separation and removal of an excess length of the elongate post 40 distal of the fracture zone F, thereby providing the bone anchor 10 with a significantly shorter overall height profile.

As should be appreciated, permitting the elongate post 40 to be broken off at a select axial location along the break-off region 44 eliminates the need for cutting off the excess portion of the elongate post 40 via a conventional post cutter or another type of shearing device. This in turn allows the surgeon to remove the excess portion of the elongate post 40 via application of a significantly reduced rotational input force/torque compared to the manually force applied to conventional post cutters or shearing devices, and also provides a greater degree of control and accuracy to remove the correct excess length of the elongate post 40. In the illustrated embodiment of the bone anchor 10, a rotational input force or torque of approximately 15 N-m is sufficient to initiate fracturing along a select one of the fracture initiator zones or grooves 70. The size and weight of the driver or torque application instrument used in association with the bone anchor 10 may also be significantly reduced relative to conventional post cutters or shearing devices.

As should also be appreciated, providing the break-off region 44 with several fracture initiator zones or grooves 70 allows the surgeon to break-off or fracture the elongate post 40 at a choice of multiple levels or locations, depending on how far the connector assembly 102 (and the elongate support member 104) is axially advanced or reduced along the break-off region 44 toward the bone engaging portion 12 of the bone anchor 10. Positioning of the flattened or truncated portions 62a, 62b along the break-off region 44 proximately adjacent the fracture initiator zones or grooves 70 allows the surgeon to apply the threshold amount of torque to a more precise/accurate location, which will in turn initiate fracturing along the desired fracture initiator zone or groove 70. A counter torque instrument may be used to grasp the connector assembly 102, the elongate support member 104, and/or the head 22 of the bone engaging portion 12 to provide a countering torque in opposition to the rotational force/torque applied to the break-off region 44 via the driver or torque application instrument.

It should be understood that any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. A bone anchor, comprising:
a bone engaging portion adapted for anchoring to bone; and
an elongate post portion extending along a longitudinal axis and including:
a proximal end region connected to said bone engaging portion;
a break-off region extending axially from said proximal end region; and
a guide region extending axially from said break-off region and configured for sliding engagement with an implant; and
wherein said break-off region includes a pre-defined fracture initiator zone to facilitate initiation of a fracture therealong upon application of a sufficient torsional force onto said break-off region and removal of a length of said elongate post portion distal of said fracture.

2. The bone anchor of claim 1, wherein said break-off region includes a plurality of said pre-defined fracture initiator zones axially offset from one another along said longitudinal axis to facilitate initiation of said fracture along a select one of said pre-defined fracture initiator zones upon application of said torsional force onto said break-off region.

3. The bone anchor of claim 2, wherein said break-off region includes at least three of said pre-defined fracture initiator zones axially offset from one another along said longitudinal axis.

4. The bone anchor of claim 1, wherein said pre-defined fracture initiator zones provide regions of reduced strength along said break-off region relative to adjacent portions of said break-off region.

5. The bone anchor of claim 4, wherein said regions of reduced strength each have a reduced transverse cross section relative to said adjacent portions of said break-off region to thereby define said pre-defined fracture initiator zones.

6. The bone anchor of claim 5, wherein said reduced transverse cross section is defined by an annular groove extending about said longitudinal axis and positioned between said adjacent portions of said break-off region.

7. The bone anchor of claim 5, wherein said regions of reduced strength are each defined by a localized reduction in material thickness of said break-off region between said adjacent portions of said break-off region.

8. The bone anchor of claim 1, wherein said pre-defined fracture initiator zone comprises an annular groove extending about said longitudinal axis.

9. The bone anchor of claim 8, wherein said annular groove extends entirely about said break-Off region of said elongate post portion.

10. The bone anchor of claim 8, wherein said annular groove has a semi-circular bottom surface and generally planar side surfaces extending from said semi-circular bottom surface to an exterior surface of said break-off region.

11. The bone anchor of claim 10, wherein said generally planar side surfaces outwardly taper away from one another toward said exterior surface of said break-off region.

12. The bone anchor of claim 1, wherein said break-off region includes one or more tool engagement features positioned proximately adjacent said pre-defined fracture initiator zone, said tool engagement features configured for engagement by a torque application instrument to apply said sufficient torsional force onto said break-off region to facilitate said initiation of said fracture along said pre-defined fracture initiator zone.

13. The bone anchor of claim 12, wherein said tool engagement features comprise a non-circular transverse outer cross section defined by said break-off region.

14. The bone anchor of claim 12, wherein said tool engagement features comprise a pair of truncated and substantially planar outer surfaces defined along opposite sides of said break-off region.

15. The bone anchor of claim 14, wherein said pre-defined fracture initiator zone comprises an annular groove extending about said longitudinal axis, said annular groove extending entirely about said break-off region including across said truncated and substantially planar outer surfaces.

16. The bone anchor of claim 1, wherein said elongate post portion defines a central passage extending entirely therethrough and arranged along said longitudinal axis.

17. The bone anchor of claim 1, wherein said distal guide region includes an externally threaded portion.

18. The bone anchor of claim 1, wherein said bone engaging portion has a first overall axial length, said elongate post portion having a second overall axial length that is greater than said first overall axial length of said bone engaging portion.

19. The bone anchor of claim 18, wherein said second overall axial length of said elongate post portion is at least twice as long as said first overall axial length of said bone engaging portion.

20. The bone anchor of claim 1, wherein said proximal end region of said elongate post portion is pivotally connected to said bone engaging portion to thereby provide pivotal movement of said elongate post portion relative to said bone engaging portion about a pivot axis arranged transverse to said longitudinal axis.

21. The bone anchor of claim 20, wherein said proximal end region of said elongate post portion is pivotally connected to said bone engaging portion by a pivot pin extending along said pivot axis.

22. The bone anchor of claim 20, wherein said bone engaging portion includes a head defining a channel therein, said proximal end region of said elongate post portion pivotally engaged within said channel to thereby provide said pivotal movement of said elongate post portion relative to said bone engaging portion about said pivot axis.

23. The bone anchor of claim 22, wherein said head includes a concave bottom surface defining a bottom of said channel, said proximal end region of said elongate post portion including a convex proximal end surface matingly engaging said concave bottom surface of said head to permit said pivotal movement of said elongate post portion relative to said bone engaging portion about said pivot axis.

24. A bone anchor, comprising:
a bone engaging portion adapted for anchoring to bone; and
an elongate post portion extending along a longitudinal axis and including:
a proximal end region connected to said bone engaging portion;
a break-off region extending axially from said proximal end region; and
a guide region extending axially from said break-off region and configured for sliding engagement with an implant; and
wherein said break-off region includes a plurality of annular grooves extending about said longitudinal axis and axially offset from one another along said longitudinal axis, said annular grooves facilitating initiation of a fracture along a select one of said annular grooves upon application of a sufficient torsional force onto said break-off region and removal of a length of said elongate post portion distal of said fracture; and
wherein said break-off region further includes one or more tool engagement features positioned proximately adjacent said annular grooves, said tool engagement features configured for engagement by a torque application instrument to apply said sufficient torsional force onto said break-off region to facilitate said initiation of said fracture along said select one of said annular grooves.

25. The bone anchor of claim 24, wherein said tool engagement features comprise a pair of truncated and substantially planar outer surfaces defined along opposite sides of said break-off region.

26. The bone anchor of claim 24, wherein said bone engaging portion has a first overall axial length, said elongate post portion having a second overall axial length that is greater than said first overall axial length of said bone engaging portion.

27. The bone anchor of claim 26, wherein said second overall axial length of said elongate post portion is at least twice as long as said first overall axial length of said bone engaging portion.

28. The bone anchor of claim 24, wherein said proximal end region of said elongate post portion is pivotally connected to said bone engaging portion by a pivot pin extending along said pivot axis.
